(19) United States
(12) Patent Application Publication
(10) Pub. No.: US 2015/0184082 A1
(43) Pub. Date: Jul. 2, 2015

(54) SYSTEM AND METHOD FOR PRODUCING GASOLINE

(71) Applicant: MITSUBISHI HEAVY INDUSTRIES, LTD., Tokyo (JP)

(72) Inventors: Masaki Iijima, Tokyo (JP); Ryuji Yoshiyama, Tokyo (JP); Haruaki Hirayama, Tokyo (JP); Yoshio Seiki, Tokyo (JP)

(73) Assignee: MITSUBISHI HEAVY INDUSTRIES, LTD., Tokyo (JP)

(21) Appl. No.: 14/406,668

(22) PCT Filed: Jun. 19, 2013
(86) PCT No.: PCT/JP2013/066813

§ 371 Date: Dec. 9, 2014

(30) Foreign Application Priority Data
Jul. 6, 2012 (JP) 2012-152451

(51) Int. Cl. C10G 3/00; C07C 29/151; C01B 3/38; B01J 19/24; C10L 1/06

(52) U.S. Cl. CPC C10G 3/42; B01J 19/245; C10L 1/06; C01B 3/38; C07C 29/1518; C10L 2200/0492; C10L 2270/023; C10L 2290/42; C10L 2290/10; C01B 2203/0233; C01B 2203/1241; C01B 2203/061

(57) ABSTRACT

A system or method for producing gasoline from natural gas can be particularly useful in a location which is a natural gas-producing region, but in which it is difficult to obtain water suitable for use in steam reforming, for example, in a desert or at sea. A system for producing gasoline from natural gas via methanol according to the present invention includes: a steam reformer 20 for steam-reforming natural gas to produce reformed gas; a methanol synthesis apparatus 30 for synthesizing methanol from the reformed gas; and a gasoline synthesis apparatus 50 for synthesizing gasoline from the methanol, water being produced in the gasoline synthesis apparatus 50 is reused for the steam reforming in the steam reformer 20.

### TECHNICAL FIELD

The present invention relates to a system and to a method for producing gasoline, and more specifically, relates to a system and to a method for producing gasoline from natural gas via methanol.

### BACKGROUND ART

As a method for producing gasoline from natural gas, Japanese Patent Publication (B2) No. S62-041276 discloses a method in which synthesis gas is produced by treating natural gas with steam, methanol is synthesized from the synthesis gas, and gasoline is further synthesized from the methanol. In a reaction for synthesizing gasoline from methanol, a large amount of water is produced in addition to gasoline. However, no method for using such water has been conventionally researched.

### BACKGROUND LITERATURE

### Patent Literature

Patent Literature 1: Japanese Patent Publication (B2) No. S62-041276

### DISCLOSURE OF INVENTION

### Problem to be Solved by Invention

An object of the present invention is to provide a system or a method for producing gasoline in which in producing gasoline from natural gas via methanol, water produced as a result of synthesis of gasoline can be effectively used.

### Means for Solving the Problem

According to an aspect of the present invention, a system for producing gasoline from natural gas via methanol includes a steam reforming apparatus for steam-reforming natural gas by using water to produce reformed gas, a methanol synthesis apparatus for synthesizing methanol from the reformed gas produced by the steam reforming apparatus, a gasoline synthesis apparatus for producing gasoline and water from the methanol synthesized by the methanol synthesis apparatus, and a line for feeding the water produced by the gasoline synthesis apparatus to the steam reforming apparatus to use the water for the steam reforming of the natural gas.

The system according to the present invention may further include a carbon dioxide recovery apparatus for recovering carbon dioxide from a flue gas generated in the steam reforming apparatus, and a line for feeding the carbon dioxide recovered by the carbon dioxide recovery apparatus to the steam reforming apparatus.

According to another aspect of the present invention, a method for producing gasoline from natural gas via methanol includes a step of steam-reforming natural gas by using water to produce reformed gas, a step of synthesizing methanol from the reformed gas, a step of producing gasoline and water from the methanol, and a step of reusing the water produced in the gasoline synthesis for the steam reforming of the natural gas.

The method according to the present invention may further include a step of recovering carbon dioxide from a flue gas generated in the steam reforming of the natural gas, and a step of introducing the recovered carbon dioxide to the steam-reforming of the natural gas.

### Advantageous Effects of Invention

As described above, according to the present invention, a large amount of steam necessary for steam reforming of natural gas can be afforded by reusing the water produced in the gasoline synthesis for the steam reforming of natural gas. In particular, natural gas-producing regions are often in deserts and at sea, where it is difficult to obtain fresh water available for the steam reforming, and thus, it is very effective to afford the necessary and available water within the system.

### BRIEF DESCRIPTION OF DRAWINGS

FIG. 1 is a schematic diagram showing an embodiment of a system for producing gasoline from natural gas via methanol according to the present invention.

FIG. 2 is a schematic diagram showing another embodiment of a system for producing gasoline from natural gas via methanol according to the present invention.

### DESCRIPTION OF EMBODIMENTS

Embodiments of a system and a method for producing gasoline from natural gas via methanol according to the present invention will now be described with reference to the accompanying drawings.

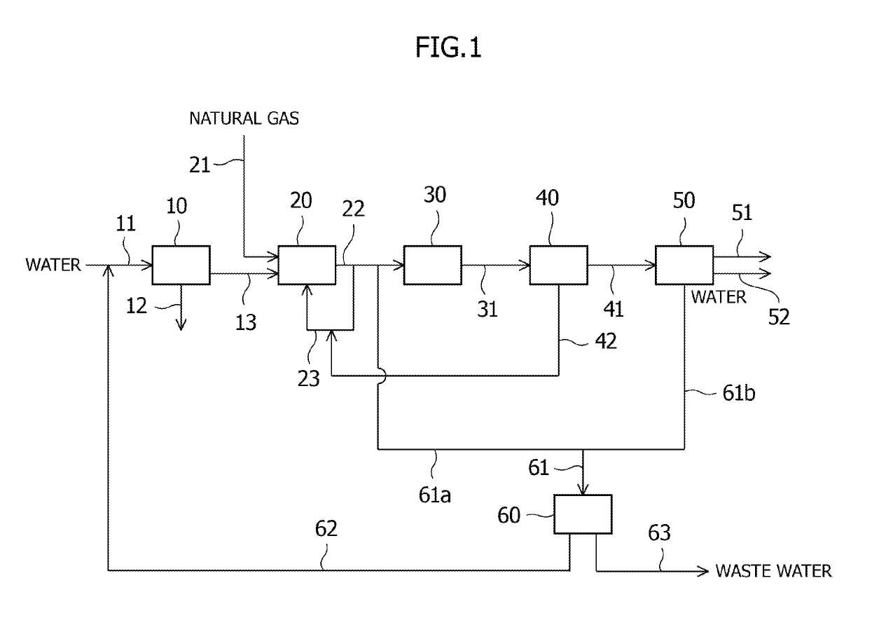

As shown in FIG. 1, a system according to the present embodiment includes a boiler 10 which generates steam, a steam reformer 20 which steam-reforms natural gas to produce reformed gas, a methanol synthesis column 30 which synthesizes methanol from the reformed gas produced by the steam reformer, a gasoline synthesis column 50 which synthesizes gasoline from the methanol synthesized by the methanol synthesis column, and a water recovery line 61 which recovers water produced in the gasoline synthesis column to reuse it in the steam reformer.

The boiler 10 is not particularly limited to a specific apparatus so long as it boils water into steam. The boiler 10 is provided with a water feed line 11 for feeding water to the boiler 10, a water discharge line 12 for discharging waste water from the boiler, and a steam feed line 13 for feeding the steam generated in the boiler to the steam reformer 20.

The steam reformer 20 includes reaction tubes (not shown) filled with a steam reforming catalyst, in which hydrogen, carbon monoxide, and carbon dioxide are produced from natural gas containing methane as the primary component by a reaction expressed by the following formula. As the steam reforming catalyst, publicly known catalysts such as a nickel-based catalyst can be used.

CH₄+H₂O→3H₂+CO (Formula 1)

A natural gas feed line 21 for feeding natural gas to the steam reformer 20 as well as the steam feed line 13 from the boiler are connected on an inlet side of the reaction tubes of the steam reformer 20. A reformed gas feed line 22 for feeding reformed gas, which contains hydrogen, carbon monoxide and carbon dioxide as the main components, to the methanol synthesis column 30 is connected on an outlet side of the reaction tubes of the steam reformer 20.

The reformed gas feed line 22 is provided with a steam return line 23 for returning water into which a part of the reformed gas in the line 22 is condensed to the steam reformer 20 as steam. Also, the reformed gas feed line 22 is provided with a water recovery line 61a for temporarily recovering the condensed water as water.

The methanol synthesis column 30 is an apparatus for synthesizing methanol from the reformed gas by a reaction expressed by the following formula.

3H₂+CO→CH₃OH+H₂ (Formula 2)

The methanol synthesis column 30 includes a methanol synthesis catalyst filled in in an inside thereof. As the methanol synthesis catalyst, publicly known catalysts such as a copper-based catalyst can be used. The reformed gas feed line 22 is connected to the methanol synthesis column 30 on an inlet side thereof A crude methanol feed line 31 for feeding crude methanol which is synthesized in the methanol synthesis column 30 to a distillation column 40 is connected to the methanol synthesis column 30 on an outlet side thereof.

The crude methanol contains water as well as methanol. The distillation column 40 is an apparatus which separates water from the crude methanol by distillation. To the distillation column 40, connected are a methanol feed line 41 for feeding purified methanol to the gasoline synthesis column 50 and a distilled water recovery line 42 for recovering the distilled water separated from methanol and feeding the recovered distilled water to the methanol synthesis column 30.

The gasoline synthesis column 50 is an apparatus which synthesizes gasoline from methanol by a reaction expressed by the following formula.

nCH₃OH→n(CH₂)+nH₂O (Formula 3)

As expressed by Formula 3, gasoline and water are produced from methanol at a molar ratio of 1:1. Note that in the synthesis of gasoline from methanol, a reaction for synthesizing gasoline from dimethyl ether (DME) occurs after completing a reaction for synthesizing DME from methanol. Accordingly, in the gasoline synthesis column 50, two types of catalysts including a DME synthesis catalyst and a gasoline synthesis catalyst are provided in two stages to gradually run the two reactions. As the DME synthesis catalyst, publicly known catalysts such as an aluminosilicate type zeolite-based catalyst can be used. In addition, for the gasoline synthesis catalyst, publicly known catalysts such as an aluminosilicate type zeolite-based catalyst can also be used.

A gasoline feed line 51 for feeding the gasoline synthesized in the gasoline synthesis column to storage facilities (not shown) is connected to the gasoline synthesis column 50. Note that in the gasoline synthesis column 50, a liquefied petroleum gas (LPG) is produced as a byproduct in addition to gasoline, and accordingly, an LPG feed line 52 may be separately connected. In addition, because a large amount of water is produced in the gasoline synthesis column 50 as expressed by Formula 3 mentioned above, a water recovery line 61b for recovering the water is connected thereto. Note that a mixture of gasoline and water is obtained in the gasoline synthesis column 50, which forms two phases including an aqueous phase and an oil phase due to the difference in their specific gravity. Accordingly, the gasoline and the water can be readily separated from each other by providing an oil-water separation device (not shown). With respect to conditions of the waste water which flows through the water recovery line 61b, the concentration of methanol is 1 wt. % or less, the concentration of ethanol is 10 wt.ppm or less, the concentration of other alcohols is 1 wt.ppm or less, and the concentration of oil contents is 1 wt. % or less, for example.

The water recovery line 61b of the gasoline synthesis column 50 is connected to a desalination apparatus 60 as well as a water recovery line 61a, which is provided at a subsequent stage of the steam reformer 20. The desalination apparatus 60 is an apparatus which removes impurities from the recovered water to allow the recovered water to be suitable for use in the boiler 10. The boiler water preferably has a composition which satisfies the standards specified in JIS B 8223-2006 “Water Conditioning for Boiler Feed Water and Boiler Water”. The following table shows the standards for the compositions.

TABLE 1

| Cate- | Normal operation | Over 5 and | | | Over 7.5 and | | Over 10 and | | | Over 15 and | | |
|---|---|---|---|---|---|---|---|---|---|---|---|---|
| gory | pressure (Mpa) | less than 7.5 | | | less than 10 | | less than 15 | | | less than 20 | | |
| | Evaporation rate on | — | | | — | | — | | | — | | |
| | heating surface | | | | | | | | | | | |
| | [kg/(m² · h)] | | | | | | | | | | | |
| | Type of makeup water | Ion exchange | | | Ion exchange | | Ion exchange | | | Ion exchange | | |
| | | water (¹⁶) | | | water (¹⁶) | | water (¹⁶) | | | water (¹⁶) | | |
| Water | Process method | — | | | — | | — | | Oxygen- | — | | Oxygen- |
| to be | | | | | | | | | ated | | | ated |
| fed | pH (at 25° C.) | 8.5 to | | | 8.5 to | | 8.5 to | | 8.0 to | 8.5 to | | 8.0 to |
| | | 9.7 (¹⁷) | | | 9.7 (¹⁷) | | 9.7 (¹⁷) | | 9.3 | 9.7 (¹⁷) | | 9.3 |
| | Electrical conductivity | — | | | — | | 0.05 or | | 0.02 or | 0.05 or | | 0.02 or |
| | (mS/m) (¹⁸) (at 25° C.) | | | | | | less | | less (¹⁹) | less | | less (¹⁹) |
| | Hardness (mgCaCo₃/L) | Not | | | Not | | Not | | Not | Not | | Not |
| | | detected (²⁰) | | | detected (²⁰) | | detected (²⁰) | | detected (²⁰) | detected (²⁰) | | detected (²⁰) |
| | Oils and Fats (mg/L) (⁹) | (¹⁰) | | | (¹⁰) | | (¹⁰) | | (¹⁰) | (¹⁰) | | (¹⁰) |
| | Dissolved oxygen | 7 or | | | 7 or | | 7 or | | 20-200 | 7 or | | 20-200 |
| | (μgO/L) | less | | | less | | less | | | less | | |
| | Iron (μgFe/L) | 50 or | | | 30 or | | 30 or | | 5 or | 20 or | | 5 or |
| | | less | | | less (²¹) | | less (²¹) | | less (²³) | less (²²) | | less (²³) |
| | Copper (μgCu/L) | 30 or | | | 20 or | | 10 or | | 10 or | 5 or | | 5 or |
| | | less | | | less | | less | | less | less | | less |
| | Hydrazine | 10 or | | | 10 or | | 10 or | | — | 10 or | | — |
| | (μgN₂H₄/L) (¹⁵) | more | | | more | | more | | | more | | |
| Boiler | Treatment method | Alkali | Phos- | Volatile | Phos- | Volatile | Phos- | Volatile | — (²⁴) | Phos- | Volatile | — (²⁴) |
| water | | treat- | phate | substance | phate | substance | phate | substance | | phate | substance | |
| | | ment | treat- | treat- | treat- | treat- | treat- | treat- | | treat- | treat- | |
| | | | ment | ment | ment | ment | ment | ment | | ment | ment | |
| | pH (at 25° C.) | 9.6 to | 9.2 to | 8.5 to | 9.0 to | 8.5 to | 8.5 to | 8.5 to | 8.0 to | 8.5 to | 8.5 to | 8.0 to |
| | | 10.5 | 10.2 | 9.7 | 10.0 | 9.7 | 9.8 | 9.7 | 9.3 (²⁴) | 9.8 | 9.7 | 9.3 (²⁴) |
| | Acid consumption | — | — | — | — | — | — | — | — | — | — | — |
| | (pH 4.8) (mgCaCO₃/L) | | | | | | | | | | | |
| | Acid consumption | — | — | — | — | — | — | — | — | — | — | — |
| | (pH 8.3) (mgCaCO₃/L) | | | | | | | | | | | |
| | Total amount of residues | — | — | — | — | — | — | — | — | — | — | — |
| | after evaporation (mg/L) | | | | | | | | | | | |
| | Electrical conductivity | 50 or | 40 or | — | 15 or | — | 6 or | — | — | 6 or | — | — |
| | (mS/m) (¹⁸) (at 25° C.) | less | less | | less | | less | | | less | | |
| | Electrical conductivity | — | — | 6 or | — | 6 or | — | 2 or | 0.3 or | — | 2 or | 0.3 or |
| | (mS/m) (at 25° C.) | | | less | | less | | less | less | | less | less |
| | Chloride ion (mgCl⁻/L) | 50 or | 50 or | 2 or | 10 or | 2 or | 2 or | 1 or | 0.05 or | 2 or | 1 or | 0.05 or |
| | | less | less | less | less | less | less | less | less (²⁵) | less | less | less (²⁵) |
| | Phosphate ion | 3 to | 3 to | (²⁶) | 2 to | (²⁶) | 0.1 to | (²⁶) | — | 0.1 to | (²⁶) | — |
| | (mgPO₄³⁻/L) (¹¹) | 10 | 10 | | 6 | | 3 | | | 3 | | |
| | Sulfite ion (mgSO₃²⁻/L) | — | — | — | — | — | — | — | — | — | — | — |
| | Hydrazine | — | — | — | — | — | — | — | — | — | — | — |
| | (μgN₂H₄/L) (¹³) | | | | | | | | | | | |
| | Silica (mgSiO₂/L) (²⁷) | 5 or | 5 or | 5 or | 2 or | 2 or | 0.3 or | 0.3 or | 0.3 or | 0.2 or | 0.2 or | 0.2 or |
| | | less | less | less | less | less | less | less | less | less | less | less |

In order to satisfy the above-described standard, the desalination apparatus 60 can be provided with activated carbon for primarily removing organic impurities, an ion exchange resin for primarily removing ionic impurities, and a degasifying drum for primarily removing gaseous contents in the fluid, and the like, for example. To the desalination apparatus 60, in order to reuse treated water treated by the desalination apparatus as steam for steam reforming, a water reuse line 62 for feeding the treated water to a water feed line 11 of the boiler 10 is connected, and also a water discharge line 63 for discharging waste water produced in the treatment by the desalination apparatus is connected.

According to the above-described configuration, at first, water is fed to the boiler 10 via the water feed line 11. Steam generated in the boiler 10 is fed to the steam reformer 20 via the steam feed line 13, and natural gas is fed to the steam reformer 20 via the natural gas feed line 21. In the steam reformer 20, the natural gas is steam-reformed by the reaction of Formula 1 mentioned above at a predetermined high temperature to be converted into reformed gas having hydrogen, carbon monoxide, and carbon dioxide as the main components. The reformed gas is fed to the methanol synthesis column 30 via the reformed gas feed line 22.

In the reformed gas feed line 22, a part of the reformed gas is returned to the steam reformer 20 via a steam return line 23 as steam to be used in a steam reforming reaction. The ratio of the steam returned via the steam return line 23 among the steam fed to the steam reformer 20 is preferably 10 to 30%, for example. In addition, the molar ratio of the steam to the methane contained in the natural gas is theoretically 1:1; however, it is preferable to feed an excess amount of steam in order to efficiently run the steam reforming reaction. For example, 2.5 to 3.5 mol of steam can be fed for 1 mol of carbon contents contained in the natural gas. In addition, in the reformed gas feed line 22, a part of the reformed gas is fed to the desalination apparatus 60 via the water recovery line 61a as water.

In the methanol synthesis column 30, methanol is synthesized from the reformed gas by the reaction of Formula 2. The methanol synthesized by the methanol synthesis column 30 is fed to the distillation column 40 via the crude methanol feed line 31 as crude methanol containing water. The methanol purified by the distillation column 40 is fed to the gasoline synthesis column 50 via the methanol feed line 41. In addition, the distilled water separated from the crude methanol in the distillation column 40 is fed to the steam reformer 20 through the steam return line 23 via the distilled water recovery line 42.

In the gasoline synthesis column 50, gasoline is synthesized from methanol by the reaction of Formula 3. The synthesized gasoline is stored in predetermined storage facilities via the gasoline feed line 51, and the LPG produced as a byproduct is stored in the predetermined storage facilities via the LPG feed line 52. In addition, the water produced by the gasoline synthesis column 50 is fed to the desalination apparatus 60 via the water recovery line 61b.

In the desalination apparatus 60, a treatment for removing impurities from the water recovered via the water recovery line 61 is performed until the water becomes suitable for use in the boiler 10. The treated water is fed to the boiler 10 through the water feed line 11 via the water recovery line 61. In addition, the waste water produced in the desalination apparatus 60 is discharged via the water discharge line 62.

As described above, in the method for producing gasoline from natural gas via methanol, the amount of input water is equal to the amount of output water as expressed by Formulas 1 to 3 mentioned above, and the amount of water is balanced by reusing the water produced in the gasoline synthesis column 50 as the water for the steam reforming by the steam reformer 20. Accordingly, it is difficult to obtain fresh water which can be used for steam reforming in locations in a desert or at sea that are production fields of natural gas; however, according to the present invention, water which can be used for steam reforming can be easily afforded within the system.

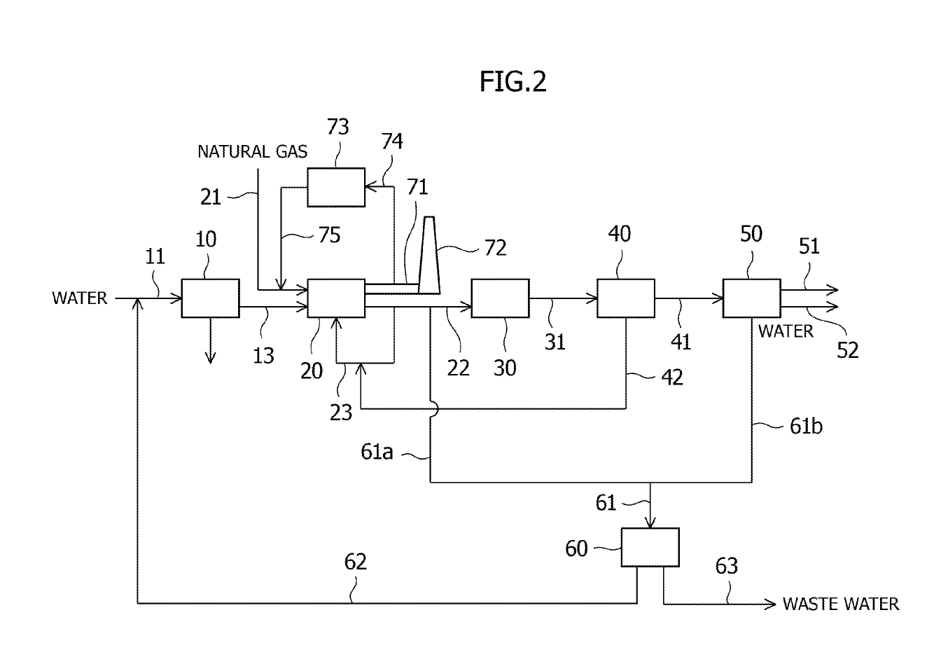

Next, another embodiment, illustrated in FIG. 2, will be described. In this embodiment, elements that are the same as those of the system illustrated in FIG. 1 are designated by the same reference numerals, and detailed descriptions thereof will not be repeated. In the system according to the present embodiment, an element for reusing a flue gas from the steam reformer 20 is provided in addition to the configuration of the system illustrated in FIG. 1.

As shown in FIG. 2, the steam reformer 20 is further provided with a flue gas path 71 for releasing flue gasses from a combustion apparatus (not shown) which heats the steam reformer 20 to a predetermined temperature to carry out steam reforming out of a stack 72, a flue gas extraction line 74 for extracting a part of the gas from the flue gas path 71, a CO₂ recovery apparatus 73 which recovers carbon dioxide from the extracted gas, and a CO₂ reuse line 75 for adding the recovered carbon dioxide to the gas flowing in the natural gas feed line 21.

The CO₂ recovery apparatus 73 is not particularly limited to a specific apparatus so long as it is capable of separating and recovering carbon dioxide from combustion flue gas. For example, an apparatus which uses a carbon dioxide absorbing liquid may be used as the CO₂ recovery apparatus 73.

According to the above-described configuration, the flue gas is discharged from the combustion apparatus (not shown) for heating the steam reformer 20 to a predetermined temperature via the flue gas path 71. A part of the flue gas is fed to the CO₂ recovery apparatus 73 via the flue gas extraction line 74, and carbon dioxide is separated and recovered there. In addition, the recovered carbon dioxide is fed to the steam reformer 20 through the natural gas feed line 21 via the CO₂ reuse line 75. A part of the carbon dioxide recovered in the above-described manner is converted into carbon monoxide in the steam reformer 20, and the carbon monoxide is fed to the methanol synthesis column 30. In the methanol synthesis column 30, a reaction expressed by Formula 4 shown below is run due to the presence of the carbon dioxide as well as the reaction expressed by Formula 2.

3H₂ +CO→CH₃OH+H₂ (Formula 2)

H₂+CO₂→CH₃OH+H₂O (Formula 4)

As described above, in the methanol synthesis column 30, surplus hydrogen reacts with carbon dioxide to produce methanol and water. More specifically, water can be produced in an amount larger than that in the embodiment illustrated in FIG. 1. The water is separated by the distillation column 40 from crude methanol to be reused by the steam reformer 20 via the distilled water recovery line 42. In addition, because the amount of output water is greater than the amount of input water in the present embodiment, the increased water can not only be reused in the steam reformer 20 but also be reused as makeup water in the boiler 10.

The present invention is not limited to the embodiments described above. For example, in FIGS. 1 and 2, the distillation column 40 is disposed between the methanol synthesis column 30 and the gasoline synthesis column 50; however, the methanol may contain water because water is produced by the synthesis of gasoline as a byproduct by the reaction expressed by Formula 3, and accordingly, the crude methanol obtained by the methanol synthesis column 30 may be fed to the gasoline synthesis column 50 via the crude methanol feed line 22 without distilling the same.

### EXAMPLES

Simulation of water balance was carried out for the embodiment illustrated in FIG. 1. The results are shown in Table 2. Note that the simulation was carried out for the case in which the daily production of methanol is 2,500 t. For the condition of the material, natural gas was used.

TABLE 2

| | Flow rate of water (ton/h) |
|---|---|
| Water feed line 11 | >152.2 |
| Steam reformer 20 | 206.5 |
| Steam feed line 13 | 152.2 |
| Steam return line 23 | 54.3 |
| Distilled water recovery line 42 | 23.6 |
| Water recovery line 61 | 150.8 |
| Water recovery line 61a | 88.2 |
| Water recovery line 61b | 62.6 |

As shown in Table 2, it was necessary to feed an excessive amount of steam to the steam reformer compared to the amount of feed of the natural gas, and it was necessary to feed the steam of about 200 ton/h (in total of the steam fed via the steam feed line and the steam return line). For about 25% of the steam, the steam discharged from the steam reformer was returned, and for the rest of the steam, the water produced in the gasoline synthesis column was recovered and used, and accordingly, almost all the steam to be fed to the steam reformer was afforded within the system. Note that the daily production of gasoline was 8,135 barrels and the daily production of LPG was 122 tons.

Next, simulation was carried out for the amount of increase of water in the system provided with the CO₂ recovery apparatus for the embodiment illustrated in FIG. 2. In the simulation, the daily production of methanol was 2,500 tons and natural gas was used as the material just as in the above-described simulation. As a result, the flow rate of the carbon dioxide added from the CO₂ recovery apparatus to the steam reformer was 42.6 ton/h, and the flow rate of the water obtained in the methanol synthesis column by the reaction of Formula 4 was 17.4 ton/h. In the methanol synthesis column, 31.0 ton/h of methanol is produced together with water, and accordingly, methanol which is the material is increased in the gasoline synthesis column by this amount. As a result, the amount of gasoline increases and also the water is increased by 17.4 ton/h. Accordingly, by adding 42.6 ton/h of carbon dioxide, the water is increased by 34.8 ton/h. This increased amount is sufficient for the makeup water for the boiler.

### DESCRIPTION OF REFERENCE NUMERALS

10: Boiler
11: Water feed line
12: Water discharge line
13: Steam feed line
20: Steam reformer
21: Natural gas feed line
22: Reformed gas feed line
23: Steam return line
30: Methanol synthesis column
31: Crude methanol feed line
40: Distillation column
41: Methanol feed line
42: Distilled water recovery line
50: Gasoline synthesis column
51: Gasoline feed line
52: LPG feed line
60: Desalination apparatus
61 Water recovery line
62: Water reuse line
63: Water discharge line
71: Flue gas path
72: Stack
73: CO₂ recovery apparatus
74: Flue gas extraction line
75: CO₂ reuse line

1-4. (canceled)

5. A system for producing gasoline from natural gas via methanol, comprising:
a steam reforming apparatus for steam reforming the natural gas by using water to produce reformed gas;
a methanol synthesis apparatus for synthesizing methanol from the reformed gas produced by the steam reforming apparatus;
a gasoline synthesis apparatus for producing gasoline and water from the methanol synthesized by the methanol synthesis apparatus;
a line for feeding the water produced by the gasoline synthesis apparatus to the steam reforming apparatus to use the water for steam reforming of the natural gas;
a carbon dioxide recovery apparatus for recovering carbon dioxide from a flue gas generated in the steam reforming apparatus; and
a line for feeding the carbon dioxide recovered by the carbon dioxide recovery apparatus to the steam reforming apparatus.

6. A method for producing gasoline from natural gas via methanol, comprising the steps of:
steam reforming the natural gas by using water to produce reformed gas;
synthesizing methanol from the reformed gas;
producing gasoline and water from the methanol;
reusing the water produced in the gasoline synthesis for the steam reforming of the natural gas;
recovering carbon dioxide from a flue gas generated in the steam reforming of the natural gas; and
introducing the recovered carbon dioxide to the steam reforming of the natural gas.
